(19) United States
(12) Patent Application Publication
(10) Pub. No.: US 2019/0335741 A1
(43) Pub. Date: Nov. 7, 2019

(54) ACCELERATED SCENTING DEVICE

(71) Applicant: Raymond Byron Willis, Shelbyville, TN (US)

(72) Inventor: Raymond Byron Willis, Shelbyville, TN (US)

(21) Appl. No.: 15/969,423

(22) Filed: May 2, 2018

(51) Int. Cl. A01M 31/00; A61L 9/12

(52) U.S. Cl. CPC A01M 31/008; A61L 9/122

(57) ABSTRACT

A scenting device adapted to accelerate a selectable scent along a straight, linear blow-through unit design is provided. The scent dispersing device has a tubular body extending from an upward opening to a downward opening, wherein a fan is disposed in the tubular body between the upward and downward openings. A scent pad is disposed in the tubular body downward of the fan, the fan adapted to selectively urge air flow from the upward opening toward the downward opening.

### BACKGROUND OF THE INVENTION

The present invention relates to accessories for the sport of hunting and, more particularly, to a device that selectively accelerates a desired scent for recreational or hunting purposes.

Recreational game hunters are always looking for an edge when hunting since their prey is usually blessed with superior sense, not least of which is a keen sense of smell that can detect threats, such as a hunter's presence, or that can smell attractive scents that lure the prey to where a hunter lays in wait.

Many current scent dispensers are merely scenting device designed for home use, but utilized in a makeshift manner in the great outdoors. Typically, they draw air in from the sides of their housing and blow it out the front thereof, which is sub-optimally responsive, an ineffective use of the motor, and causes a wasteful battery drain.

As can be seen, there is a need for a scenting device that is adapted for outdoor use, particularly for recreational game hunting, wherein the scenting device accelerates a selectable scent along a linear air flow. Such a straight, linear blow-through unit design accelerates air flow at three times the rate as compared to scenting units drawing air through the sides of their housing, making optimal use of the motor, and not inefficiently draining the batteries, so that user can hunt longer with the present invention as opposed to currently available hunting scenting devices.

The present invention enables hunters to quickly change the ambient scent of their surroundings to produce a different desired scent from a palette of selectable scents, adapting at a moment's notice to the inherent unpredictability that is outdoor game hunting.

As a result, the scenting device can be used by hunters in a variety of ways, including but not limited to covering their scent and emitting an attractive scent to lure the prey to the hunter.

### SUMMARY OF THE INVENTION

In one aspect of the present invention, a scent dispensing device includes a tubular body extending from an upward opening to a diametrically opposed downward opening; a fan disposed in the tubular body between the upward and downward openings, the fan configured to urge air flow from the upward opening tow; and a scent pad attached immediately adjacent to the downward opening.

In another aspect of the present invention, the scent dispensing device includes a tubular body extending from an upward opening to a diametrically opposed downward opening; a fan disposed in the tubular body between the upward and downward openings, the fan configured to urge air flow from the upward opening tow; a downward grate removably secured along the downward opening; a scent pad removably disposed along an upper surface of the downward grate; at least one pair of diametrically opposing holes provided through the tubular body just inward of the downward opening; a peg slidably received through each pair of diametrically opposing holes so as to outwardly protrude from each hole; an elastic element extending between opposing ends of each peg so as to further secure said downward grate; and a control circuitry configured to selectively control the rotational speed of the fan.

These and other features, aspects and advantages of the present invention will become better understood with reference to the following drawings, description and claims.

### BRIEF DESCRIPTION OF THE DRAWINGS

FIG. 1 is a front perspective view of an exemplary embodiment of the present invention;

FIG. 2 is a front perspective view of an exemplary embodiment of the present invention;

FIG. 3 is an exploded perspective view of an exemplary embodiment of the present invention;

FIG. 4 is a cross-sectional view of an exemplary embodiment of the present invention; and

FIG. 5 is an electronic schematic view of an exemplary embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a scenting device adapted to accelerate a selectable scent along a straight, linear blow-through unit design. The scent dispersing device has a tubular body extending from an upward opening to a downward opening, wherein a fan is disposed in the tubular body between the upward and downward openings. A scent pad is disposed in the tubular body downward of the fan, the fan adapted to selectively urge air flow from the upward opening toward the downward opening.

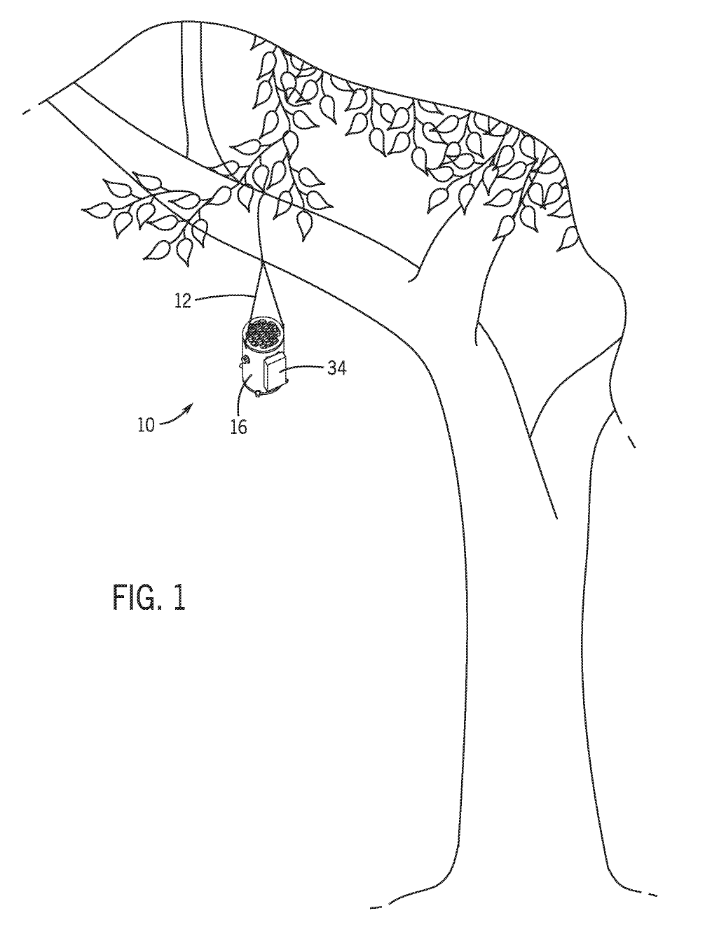

Referring to FIGS. 1 through 5, the present invention may include a scent dispenser device 10 adapted in a straight, linear blow-through unit design. Such a design embodies a tubular housing 16 extending from an upward end 51 to a downward end 52, wherein the tubular housing 16 is disposed a scent pad 28 downward of a fan 26 so that the fan urges air flow from the upward end 51 past the scent pad 28 out of the downward end 52. The tubular housing 16 may be made from various plasticized material and extend for approximately three to six inches with the fan 26 disposed generally midpoint between the upward and downward ends 51 and 52.

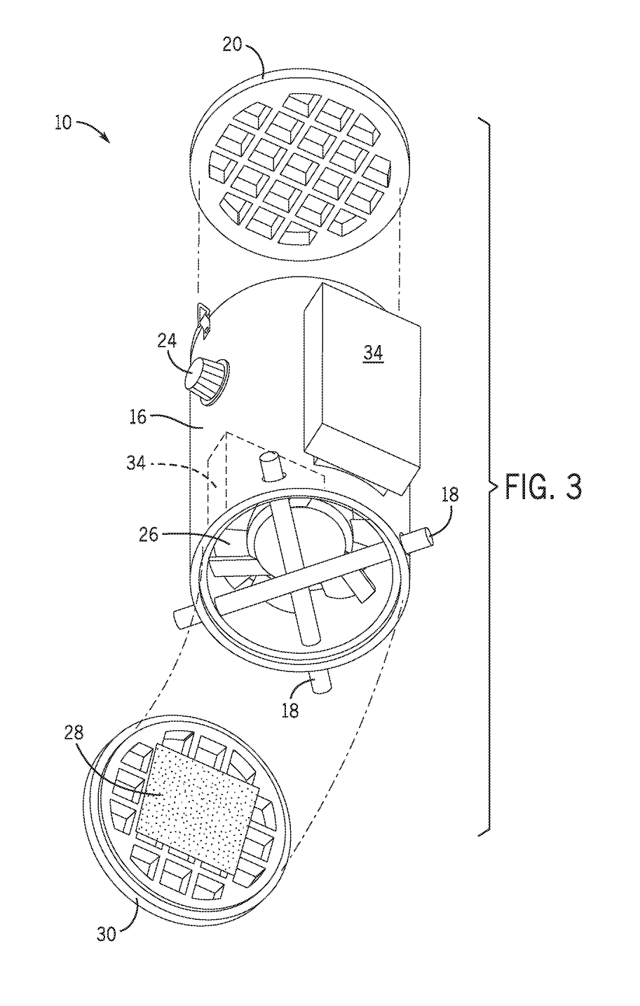

Removably attached to the upward end 51 may be an upward grate 20, while removably attached to the downward end 52 may be a downward grate 30. Each grate 20 and 30 may have a plurality of apertures for allowing air to pass therethrough. The scent pad 28 may be removably lain on the inward surface of the downward grate 30, as illustrated in FIG. 3. Thereby, a user may replace a first scent pad 28 with a second scent pad 28 when wanting to change the scent emitted through the downward end 52 of the present invention. In certain embodiments, the user may lay the second scent pad 28 on top of the first scent pad 28 to create selectively blended scents.

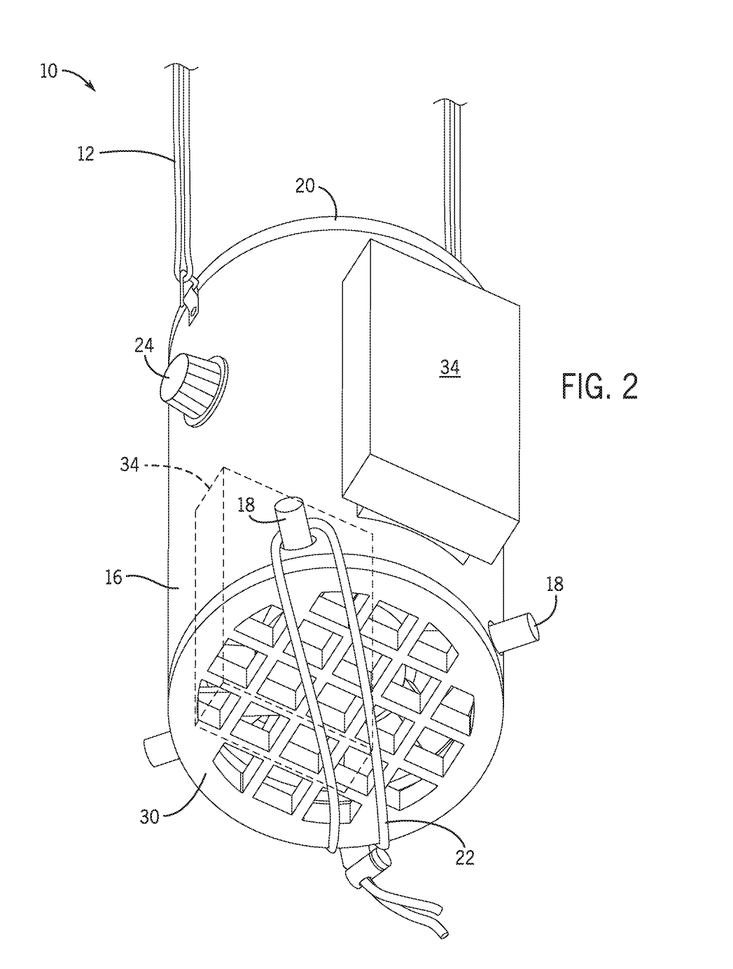

Diametrically opposing holes 17 may be disposed through the tubular housing 16 just inward of the downward end 52. Slidably received through each set of diametrically opposing holes 17 may be a peg 18. An elastic element 22 may be stretched from around opposing ends of each peg 18 protruding trough the respective holes 17 in order to secure the downward grate 30 against the downward end 52, as illustrated in FIG. 2.

In certain embodiments, the downward grate 30 (and related peg 18, holes 17, and elastic element 22) are not required and thus not present, rather the scent pad 28 is removably attached along the downward end 52 through conventional connectors (not shown).

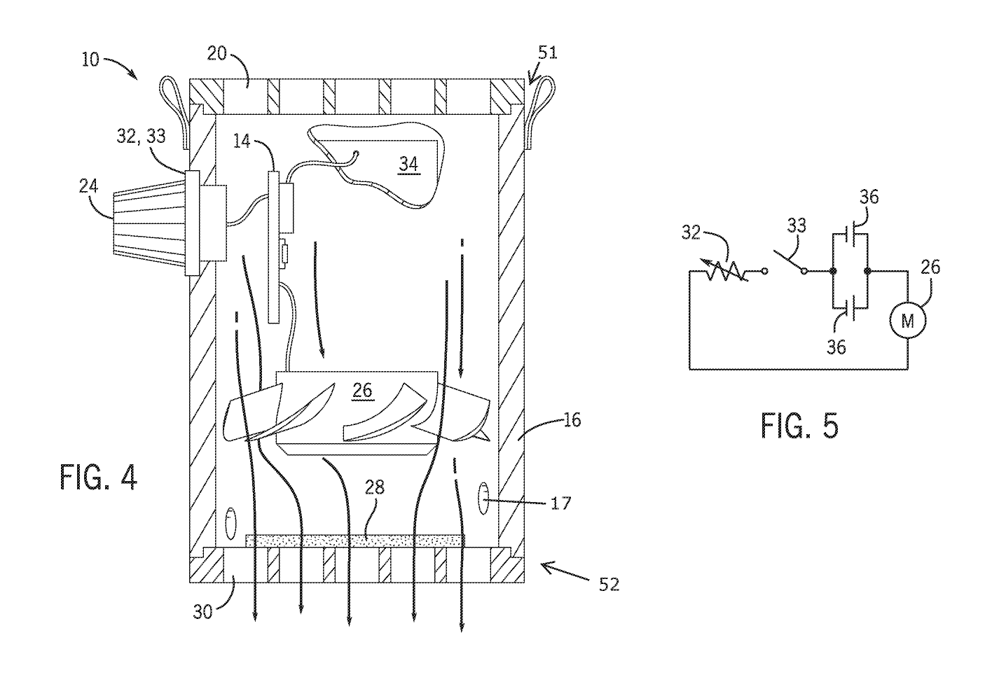

A control knob 24 may be outwardly disposed along the tubular housing 16, as illustrated in FIG. 4. The control knob 24 may be operatively associated with a control circuitry 14 coupled to a power source 36 and the fan 26. The control circuitry 14 may include electronics 32 and 33, such as resistors, transistors, switches, and the like to facilitate selective control over the speed of the fan 26, in certain embodiments, through manipulation of the control knob 24. In other embodiments, the control circuitry 14 may be operatively associated with a smart phone application. The power source(s) 36 may be rechargeable batteries housed in a batter case 34.

It should be understood by those skilled in the art that the use of directional terms such as upward, downward, and the like are used in relation to the illustrative embodiments as they are depicted in the figures, the upward direction (or upper) being toward the top of the corresponding figures and a downward direction being toward the bottom of the corresponding figure.

A method of using the present invention may include the following. The dispenser device 10 disclosed above may be provided. After charging the power source 34 and placing a desired scent pad 28 immediately adjacent to the downward end 52, a user may use a suspension cord 12 to hang said dispenser device 10 from a supporting object, such as a tree limb. By manipulating the control knob 24 the user may selectively control the rate of air flow urged through the opposing upward and downward ends 51 and 52 to desired rotational speed.

The user may be a hunter interested in covering their scent from their prey by placing the dispenser device 10 above them and surrounding themselves with a non-threatening (to their prey) scents. Alternatively, an attractant scent (from another scent pad 28) can be used by the hunter to attract their desired prey. In other words, the dispenser device 10 is made by an outdoors man for outdoors men and women.

Additionally, the dispenser device 10 can be used for aromatherapy, keep bugs out of tent while camping, help dry out a damp boot or wet sock from fishing.

It should be understood, of course, that the foregoing relates to exemplary embodiments of the invention and that modifications may be made without departing from the spirit and scope of the invention as set forth in the following claims.

What is claimed is:

1. A scent dispensing device, comprising:
a tubular body extending from an upward opening to a diametrically opposed downward opening;
a fan disposed in the tubular body between the upward and downward openings, the fan configured to urge air flow from the upward opening tow; and
a scent pad attached immediately adjacent to the downward opening.

2. The scent dispensing device of claim 1, further comprising:
a downward grate removably secured along the downward opening.

3. The scent dispensing device of claim 1, further comprising:
the scent pad disposed along an upper surface of the downward grate.

4. The scent dispensing device of claim 2, further comprising:
at least one pair of diametrically opposing holes provided through the tubular body just inward of the downward opening;
a peg slidably received through each pair of diametrically opposing holes so as to outwardly protrude from each hole; and
an elastic element extending between opposing ends of each peg so as to further secure said downward grate.

5. The scent dispensing device of claim 1, further comprising:
a control circuitry configured to selectively control the rotational speed of the fan.

6. The scent dispensing device of claim 1, further comprising:
an upward grate removably attachable to the upward opening.

7. A scent dispensing device, comprising:
a tubular body extending from an upward opening to a diametrically opposed downward opening;
a fan disposed in the tubular body between the upward and downward openings, the fan configured to urge air flow from the upward opening tow;
a downward grate removably secured along the downward opening;
a scent pad removably disposed along an upper surface of the downward grate;
at least one pair of diametrically opposing holes provided through the tubular body just inward of the downward opening;
a peg slidably received through each pair of diametrically opposing holes so as to outwardly protrude from each hole;
an elastic element extending between opposing ends of each peg so as to further secure said downward grate; and
a control circuitry configured to selectively control the rotational speed of the fan.

8. The scent dispensing device of claim 7, further comprising:
an upward grate removably attachable to the upward opening.
